(19) United States
(12) Patent Application Publication
(10) Pub. No.: US 2025/0050137 A1
(43) Pub. Date: Feb. 13, 2025

(54) METHOD AND APPARATUS FOR PROMOTING MOTILITY OF FLAGELLAR CELLS

(71) Applicant: Monash University, Clayton, Victoria (AU)

(72) Inventors: Junyang GAI, Clayton (AU); Citsabehsan DEVENDRAN, Clayton (AU); Reza NOSRATI, Clayton (AU); Adrian NEILD, Clayton (AU)

(21) Appl. No.: 18/719,203

(22) PCT Filed: Dec. 16, 2022
(86) PCT No.: PCT/AU2022/051529

§ 371 Date: Jun. 12, 2024

(30) Foreign Application Priority Data
Dec. 17, 2021 (AU) 2021904104

(51) Int. Cl. A61N 7/00; C12N 5/076

(52) U.S. Cl. CPC A61N 7/00; C12N 5/061; A61N 2007/0004; A61N 2007/0078; C12N 2521/10

(57) ABSTRACT

An apparatus for use in promoting motility of flagellar cells includes an acoustic energy emission module configured to generate ultrasound energy, the module being configured to generate ultrasound waves within a frequency range of about 2 MHz and about 120 MHz, and an applicator module configured to direct the generated ultrasound waves to a locus of the cells for a duration of between about 5 seconds and about 35 seconds.

### FIELD OF THE INVENTION

The present invention relates to a method and apparatus for promoting flagellar motility of motile cells, and in particular, but not exclusively, to a method and apparatus for use in promoting motility of sperm cells and in the treatment of asthenozoospermia.

### BACKGROUND OF THE INVENTION

Any reference in this specification to prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

Infertility is a rising global health issue some studies suggest that impacts over 70 million couples per year. Male factor infertility typically accounts for about 50% of infertility cases, primarily mediated by deficits in sperm count and/or sperm function. Sperm motility plays a central role in both the natural fertilisation process and clinical sperm selection as one of the key parameters determining fertility potential. In natural reproduction, sperm cells must traverse a complex journey from the seminal fluid to the viscous fluids contained within the cervix, uterus, the fallopian tube, and ultimately through the highly folded and complex lumens within the tube to achieve fertilisation. However, sperm motility can be impaired through factors attributed to the modern lifestyle such as pollution, and unhealthy diets, or through endogenous genetic factors.

Assisted reproductive technologies (ART) have been developed to circumvent infertility problems. For assisted reproduction, motile sperm cells are collected via two main clinical methods, either via a swim-up approach, in which sperm swim out of a sedimented raw semen sample into a physiological buffer, or through a density gradient centrifugation method, where motile sperm are indirectly selected for via centrifugation through a density gradient. The relatively long duration (approximately 2 hours) of these clinical selection processes, increases the degree of oxidative stresses in the sperm sample which reduces sperm motility level and increases DNA fragmentation. There exists a need for a non-invasive method to restore or enhance sperm motility without introducing potential damage to sperm cell viability or DNA integrity.

Some studies have observed an increase in metabolic activity from the use of ultrasound waves on non-motile cells. However, ultrasound waves are known to cause detrimental effects on sperm cells and ultrasound therapies have been suggested in the past to reduce sperm viability as a method for contraception (see examples: Fahim et al., Ultrasound as a New Method of Male Contraception (1977); Tsuruta et al., Therapeutic ultrasound as a potential male contraceptive: power, frequency and temperature required to deplete rat testes of meiotic cells and epididymides of sperm determined using a commercially available system, Reproductive Biology and Endocrinology (2012); and Levario-Diaz et al., Effect of acoustic standing waves on cellular viability and metabolic activity, NatureResearch (2020).

A number of further studies suggest that therapeutic ultrasound can have negative effects on the targeted tissue (see examples: C. L. Tsai, W. H. Chang, T. K. Liu, Preliminary studies of duration and intensity of ultrasonic treatments on fracture repair, Chin. J. Physiol. 35, 21-26 (1992) and D. L. Miller et al.; Bioeffects Committee of the American Institute of Ultrasound in Medicine, Overview of therapeutic ultrasound applications and safety considerations, J. Ultrasound Med. 31, 623-634 (2012)).

The applicant has determined that it would be advantageous to provide a non-invasive method and/or apparatus to promote sperm motility without compromising sperm cell viability or DNA integrity. There is also a range of other cell types in which it is desirable to promote motility, wherein the present invention has application. The present invention seeks to address or at least in part alleviate one or more problems identified above, or to provide alternatives for promoting cell motility.

### SUMMARY OF THE INVENTION

According to an aspect of the invention, there is provided an apparatus for use in promoting motility of flagellar cells, comprising an acoustic energy emission module configured to generate ultrasound energy, the module being configured to generate ultrasound waves within a frequency range of about 2 MHz and about 120 MHz; and an applicator module configured to direct the generated ultrasound waves to a locus of the cells for a duration of between about 5 seconds and about 35 seconds.

In one embodiment, the acoustic energy emission module comprises two sets of interdigitate transducers configured to generate ultrasound energy in the form of standing acoustic waves.

In one embodiment, the applicator module comprises a polydimethylsiloxane fluid chamber or a fluid chamber attached with acoustic coupling medium to house the cells.

In one embodiment, the fluid chamber attached with acoustic coupling medium is a glass fluid chamber.

In one embodiment, the acoustic energy emission module is configured to generate ultrasound waves within a frequency range of about 5 MHz and about 100 MHz.

In another embodiment, the acoustic energy emission module is configured to generate ultrasound waves within a frequency range of about 10 MHz and about 50 MHZ.

In another embodiment, the acoustic energy emission module is configured to generate ultrasound waves at a frequency of about 19.28 MHZ.

In one embodiment, the applicator module is configured to direct the generated ultrasound waves for a duration of between about 15 seconds and about 25 seconds.

In another embodiment, the applicator module is configured to direct the generated ultrasound waves for a duration of about 20 seconds.

According another aspect of the present invention, there is provided a method for promoting motility of flagellar cells, comprising the steps of: generating ultrasound waves within a frequency range of about 2 MHz and about 120 MHz; and exposing the cells to the generated ultrasound waves for a duration of between about 5 seconds and about 35 seconds.

In one embodiment, the ultrasound waves are generated in the form of standing acoustic waves by an acoustic energy emission module comprises two sets of interdigitate transducers.

In one embodiment, the cells are housed in a polydimethylsiloxane fluid chamber or a fluid chamber attached with acoustic coupling medium for exposure to the generated ultrasound waves.

In one embodiment, the fluid chamber attached with acoustic coupling medium is a glass fluid chamber.

In one embodiment, the method comprises generating ultrasound waves within a frequency range of about 5 MHz and about 100 MHz.

In another embodiment, the method comprises generating ultrasound waves within a frequency range of about 10 MHz and about 50 MHz.

In another embodiment, the method comprises generating ultrasound waves at a frequency of about 19.28 MHz.

In one embodiment, the method comprises exposing the cells to the generated ultrasound waves for a duration of between about 15 seconds and about 25 seconds.

In another embodiment, the method comprises exposing the cells to the generated ultrasound waves for a duration of about 20 seconds.

In another embodiment, the locus of the cells is exposed to the generated ultrasound waves via an applicator module.

In another embodiment, the method comprises generating ultrasound waves within a frequency range of about 5 MHz and about 100 MHz.

In another embodiment, the method comprises generating ultrasound waves within a frequency range of about 10 MHz and about 50 MHz.

In another embodiment, the method comprises generating ultrasound waves at a frequency of about 19.28 MHZ.

In another embodiment of the method, the cells are mammalian or avian sperm cells.

In another embodiment of the method, the cells are human sperm cells.

In another embodiment, the method is for treating asthenozoospermia, for improving fertilisation potential of sperm cells or for determining sperm cell viability.

In another embodiment of the method, the cells are bacterial flagellar cells.

In some embodiments, an acoustic energy emission module is provided to output ultrasound at a given frequency range, input power and for a particular duration based on cellular type.

In some embodiments, improvements in metabolic activity resulting from the cells' exposure to acoustic energy emissions of the applicator module leads to an increase in mechanical motility of the cells.

In some embodiments, ultrasound waves are transmitted to the cells through an acoustic coupling medium and optionally a glass coverslip.

In some embodiments, the method further provides a computer system for administering the generation and transmission of the ultrasound wave to the cells, wherein the computer system controls variables including frequency of the ultrasound waves, magnitude of ultrasound waves and ultrasound exposure duration.

Further aspects of the present invention and further embodiments of the aspects described in the preceding paragraphs will become apparent from the following description.

### BRIEF DESCRIPTION OF THE DRAWINGS

Further aspects of the present invention and further embodiments of the aspects described in the preceding paragraphs will become apparent from the following description. The invention will now be described, by way of example only, with reference to the accompanying drawings, in which:

FIG. 1 is a schematic view of a pair of interdigital transducers (IDTs) used to generate standing surface acoustic waves (SAW) in a polydimethylsiloxane fluid chamber, which houses sperm cells;

FIG. 2 shows a series of time-lapse images demonstrating the effect of the present invention have on sperm cells before and after its implementation;

FIG. 3 shows schematic representation of swimming trajectories of sperm cells demonstrating the effect of the present invention have on sperm cells before and after its implementation;

FIG. 4 shows a series of charts comparing before and after effects of the present invention on lateral head displacement (ALH), beat cross frequency (BCF) and curvilinear velocity (VCL) of the exposed sperm cells;

FIGS. 5-8 show a series of charts illustrating the effect of ultrasound exposure on the sperm cells with altered ultrasound frequency, power level and exposure duration;

FIGS. 9-12 show a series of charts illustrating the effect of ultrasound frequency of 19.28 MHz of the present invention on various sperm motility parameters;

FIG. 13 shows a schematic diagram for measuring motility (VSL and VCL);

FIGS. 14-15 show a series of charts illustrating sperm viability and DNA fragmentation index of sperm cells exposed at various durations to the present invention;

FIG. 16-17 show a series of charts illustrating the comparative effect that embodiments of the present invention have had on the metabolic activity of sperm cells;

FIG. 18 shows a chart indicating the change of motility (VCL) in a human sperm cell sample before and after exposure to ultrasound energy in accordance with an embodiment of the present invention;

FIG. 19 shows a perspective schematic view of a portable computing device incorporating the ultrasound apparatus of the present invention;

FIG. 20 shows a flow chart diagram of the control system of the portable computing device of FIG. 19;

FIG. 21 shows a chart indicating the change of motility (VCL) in a human sperm cell sample before and after exposure to ultrasound energy using the portable device of FIG. 19;

FIG. 22 is a schematic diagram of sample preparation using the glass coverslip method in accordance with an embodiment of the present invention.

FIG. 23 shows three schematic diagrams of different cell housing methods used for preparing the cell samples for ultrasound application, including the PDMS method (top), glass coverslip method (middle) and no medium control group (bottom);

FIG. 24 shows a chart indicating the relative post-exposure performances of the tested sample preparation methods of FIG. 23, the result indicates the improved effects of the treatment by using a glass coverslip; and

FIG. 25 shows a schematic diagram of glass fluid chamber housing method used for preparing the cell samples for ultrasound application.

### DETAILED DESCRIPTION

While aspects of the present invention will be described below for use in combination with each other in the preferred embodiments of the present invention, it is to be understood by a skilled person that some aspects of the present invention are equally suitable for use as standalone inventions that can be individually incorporated into apparatus and methods for use with other types of motile cells not specifically described herein.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The word “about” or “approximately” when used in relation to a stated reference point for a quality, level, value, number, frequency, percentage, dimension, location, size, amount, weight or length may be understood to indicate that the reference point is capable of variation, and that the term may encompass proximal qualities on either side of the reference point. In some embodiments, the word “about” may indicate that a reference point may vary by as much as 30 percent.

As used herein, the word “substantially” may be used merely to indicate an intention that the term it qualifies should not be read too literally and that the word could mean “sufficiently”, “mostly” or “near enough” for the patentee's purposes.

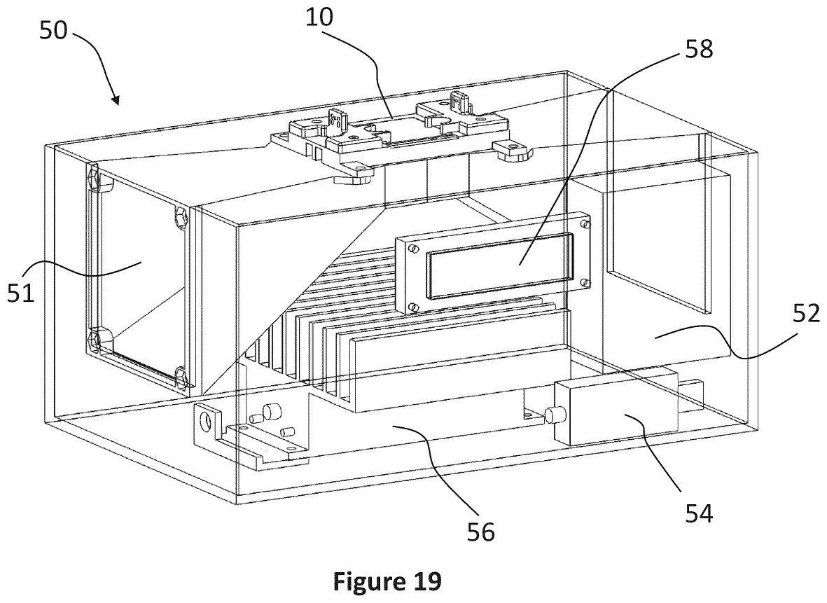

Preferred embodiments of the present invention seek to provide an improved method and/or apparatus for promoting the motility of flagellar cells, including sperm cells, and for the treatment of asthenozoospermia by exposing the cells to ultrasound waves generated and administered within selected operating ranges in relation to a combination of ultrasound frequency and exposure duration parameters. Embodiments of the present invention are defined by these operating ranges, which have been selected for their surprising and synergistic effects on enhancing the motility of motile flagellar cells. Embodiments of the present invention also seek to provide suitable methods for coupling cell samples to an ultrasound apparatus. In some embodiments, the ultrasound apparatus is provided on a platform as seen in FIG. 19, preferably portable, that is suitable for carrying out the methods described herein. The platform may be wholly embodied in a portable computer system unit, complete with power, control, display and therapy planning modules, that is designed to be used by laboratory technicians. The computing modules may comprise information about the operating conditions including, acoustic frequency, acoustic power magnitude, and acoustic exposure time.

The methods of the present invention are applicable generally to flagellar cells. Such cells comprise a flagella, which is a dynamic, whip-like tail or projection that moves through the lengthening and shortening of actin fibers. It is through the movement of the flagella, often referred to as “beating” that cells including a flagella (that is flagellar cells) are motile. The term “motile” in this context means that the flagellar cells are capable of movement and/or of moving the environment that surrounds them.

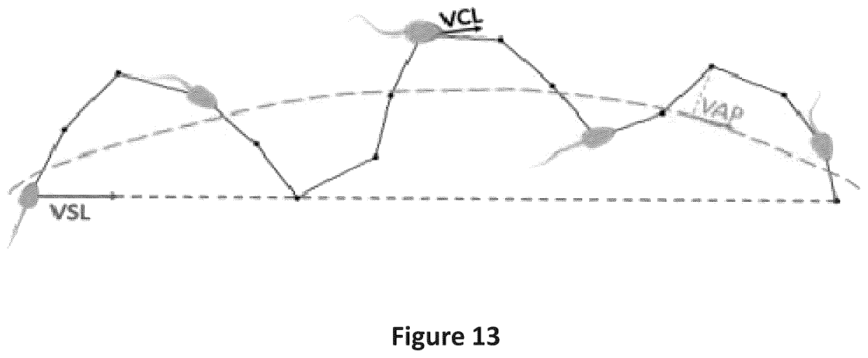

The motility of flagellar cells (also referred to herein as motile cells and cells) can readily be observed under microscopic magnification and several measures of motility are conventionally used with reference to FIG. 13, including determining average swimming velocity along an instantaneous trajectory (also known as curvilinear velocity (ACL) and instantaneous velocity), with the benefit of fluorescent labelling and time-lapse photography under microscopy. The lateral head displacement (ALH) and beat cross frequency (BCF) of flagellar cells can also be measured, as further described under the heading “sperm motility analysis” in the experimental section below.

In addition to the actual swimming velocity of the cells (VCL), other parameters also describe flagellar cell motion. Motility parameters also include the percentage of cells exhibiting swimming behaviour in the sample and the normality of cell movement, the latter being characterized by average path velocity (VAP: swimming velocity along the time-averaged trajectory), straight line velocity (VSL: time average velocity along the straight line between the start and final detection points), and linearity (LIN=VSL/VCL), as also further described under the heading “sperm motility analysis” in the experimental section below.

The exposure of flagellar cells to ultrasound energy according to the present invention has been shown by the present inventors to promote motility of the motile flagellar cells. By this, it is meant that flagella movement is initiated in cells that are stagnant (where substantially no flagella movement is detected) or is increased in cells that are already to some extent motile. For example, the exposure to ultrasound energy may initiate flagella movement in a population of flagellar cells that appear to be stagnant in at least about 2%, 5%, 8%, 10%, 15%, 20%, 30%, 40% or 50%, 60%, 70%, 80%, 90% or more of the cells. Similarly, in cells that are already motile the VCL of the cells may increase by at least about 2%, 5%, 8%, 10%, 15%, 20%, 30%, 40%, 50%, 60%, 70%, 80%, 90% or more.

The motile cells that may be subject to ultrasound energy exposure to promote motility include, for example, bacterial, reptilian, piscine, avian, marsupial and mammalian flagellar cells. For example, the activity of certain bacterial flagellar cells that ferment or otherwise process particular materials can be enhanced by increasing motility of these cells. Specific examples include species of such Marinobacter, M. as alkaliphilus, M. arcticus, M. hydrocarbonoclasticus, M. maritimus and M. squalenivorans, which exhibit beneficial activity in relation to degradation of hydrocarbons, and Pseudomonas aeruginosa, which has beneficial activity in relation to oil degradation, as well as sulphide and iron reducers such as Desulfovibrio vulgaris, Gallionella sp., Pseudomonas sp. and Thiobacillus sp.. It is to be appreciated that the specific operating ranges of the present invention, including the ultrasound frequency, input power and exposure duration parameters, which are selected for their surprising and synergistic effects, may vary in part according to target cell type without departing from the spirit of the invention.

A particular, reptilian, piscine, avian, marsupial and mammalian cell type of interest in the present invention is sperm cells, given that improved motility of sperm cells is significant for improving the potential for successful natural or in vitro egg fertilisation. For example, the promotion of sperm motility may be desired in the context of achieving fertilisation for the purpose of animal conservation, animal breeding programs and, in particular, for improving human fertility. In the case of non-human animals, sperm from companion animals (such as cats and dogs), agricultural animals (such as cattle, sheep, goats, pigs and poultry such as chickens, ducks, turkeys and geese), laboratory animals (such as mice, rats, rabbits and guinea pigs), captive wild animals (such as elephants, bears, monkeys, apes, lions and tigers) and farmed fish and other captive aquatic animals such as dolphins, seals and whales may be subject to the methods of the present invention for improving motility, for treatment of asthenozoospermia (low sperm motility) and for improving the potential for successful fertilisation.

In operation of methods of the invention the specified ultrasound energy will be exposed to the locus of the flagellar cells in which motility in desired to be promoted. For example, the cells may be retained in a flask, tube, reactor or the like, may be exposed to treatment in vivo within a male of female animal, may be within a tissue sample that has been surgically excised or cultured, may be within seminal ejaculate or may be within an isolated sample of cells in an appropriate isotonic media, which in each case would be considered the locus to which the ultrasound energy is applied in operation of the invention.

In one aspect of the invention, the specified ultrasound energy is applied to the locus of substantially stagnant flagellar cells, preferably sperm cells, as a means of determining if there are viable, potentially motile cells, within the cell sample. In this case, if flagellum movement can be detected in the cell sample after ultrasound exposure a measure of cell viability can be derived, for example as a percentage of cells that exhibit motility of the total cells in the sample. In this way the methods of the invention are useful as a prognostic device to determine fertilisation potential of sperm cells in a sample or derived from a particular individual.

It is possible to expose a single sample or locus of flagellar cells, especially sperm cells, to multiple rounds of ultrasound energy. However, in doing so it is desirable to allow a recovery period between each ultrasound exposure of, for example, from about 15 mins to about 3 hours, such as from about 20 mins to about 2 hours, about 30 mins to about 90 mins or about 35 mins, about 40 mins, about 45 mins, about 50, mins, about 55 mins or about 60 mins.

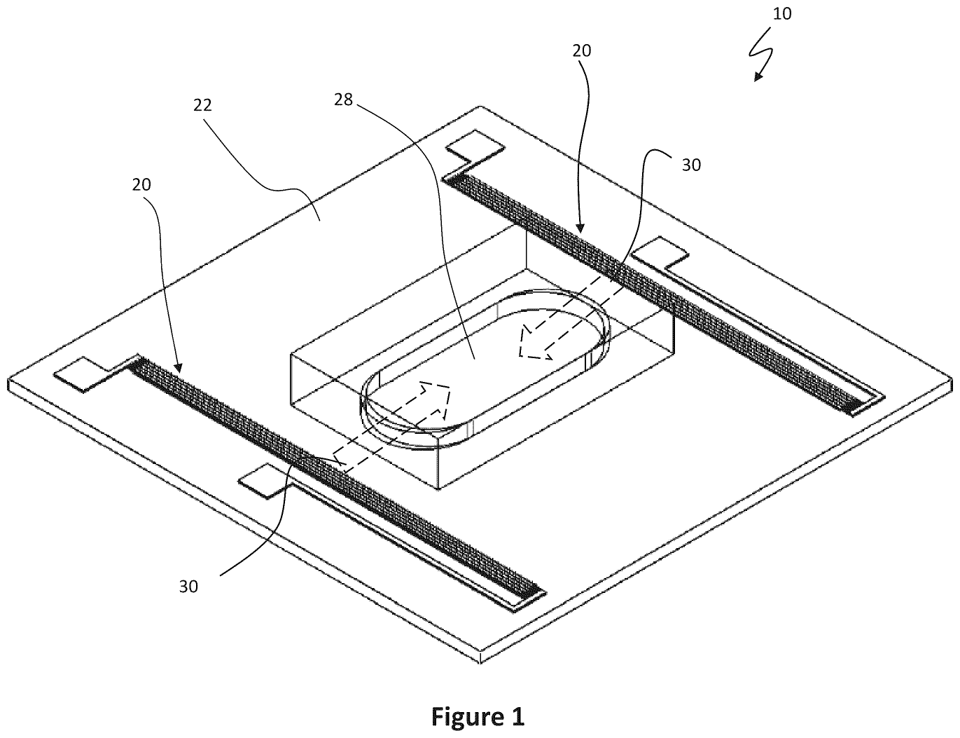

FIG. 1 shows a schematic view of an apparatus 10 in accordance with an embodiment of the present invention. The apparatus 10 comprises an acoustic energy emission module 20 having two interdigitate transducers (IDTs) configured for generating surface acoustic waves (SAW) 30 therebetween. The IDTs rest on an acoustofluidic platform 22 together with an applicator module 24 in the form of a polydimethylsiloxane (PDMS) fluid chamber 28, for containing sperm cells, located between the IDTs. In one embodiment, the IDTs are manufactured using standard UV-photolithography techniques on a suitable substrate and the IDTs are capable of generating ultrasound waves in one or more frequencies. During use, motile cells such as sperm cells located within the PDMS fluid chamber is exposed to SAW ultrasound energy 30 generated by the IDTs in accordance with a combination of specifically-defined operating conditions, including ultrasound frequency, power level and exposure duration.

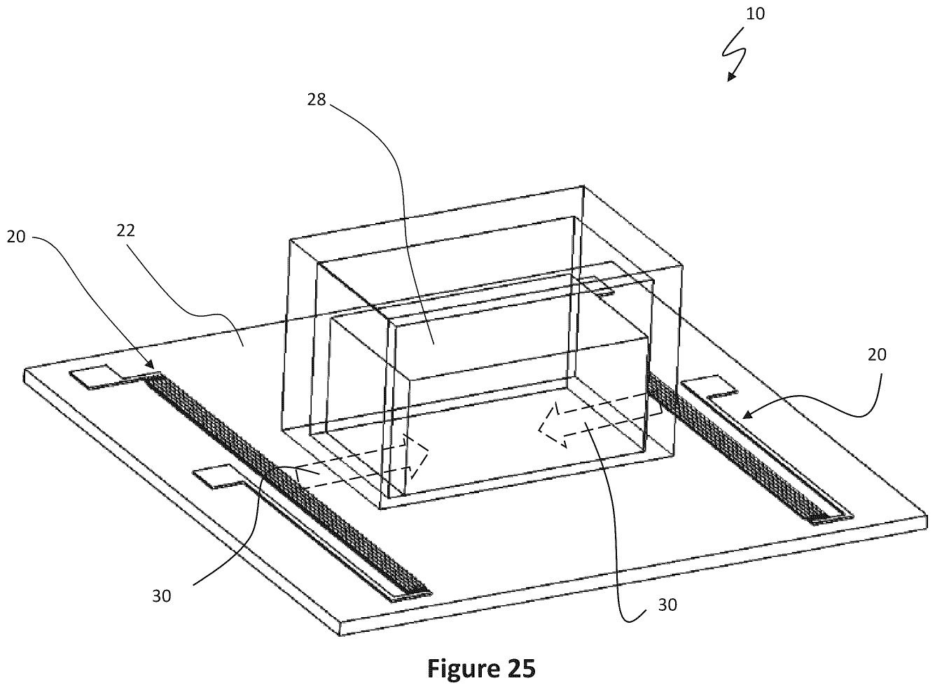

The fluid chamber 28 attached on the apparatus 10 may be customised or personalised according to the volume, viscosity, and the species of the sperm sample handled to accommodate different users and applications. Referring to FIG. 25 for example, the fluid chamber 28 is a glass fluid chamber that comprises an upwardly open structure adapted to contain a selected volume of liquid, which may facilitate the retrieval of the sample. In another example, the material of the fluid chamber 28 may be designed or selected to enhance the efficacy of the treatment. The housing assembly of the chamber 28 is provided for improved efficacy for acoustic treatment with convenience of operation and efficiency of power transmission. In some embodiments, the sample of sperm cells is provided in the fluid chamber 28 in an acoustic coupling medium 40. Non-limiting examples of acoustic coupling medium 40 include water, mineral oil, white petrolatum, gel (KY, EMS, Aquasonic, JPM, Physiomed, SKF, Biofreeze), dry couplant and semi-dry acoustic couplants (polymers).

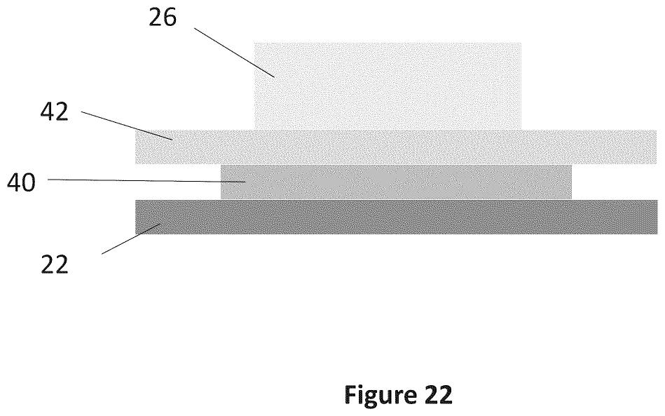

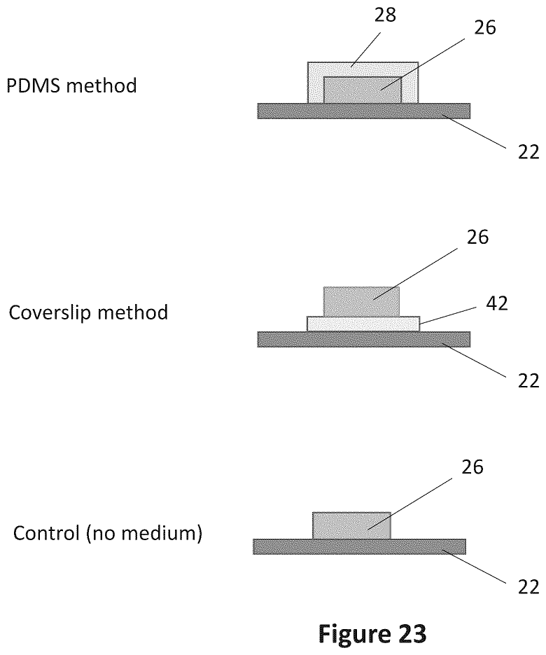

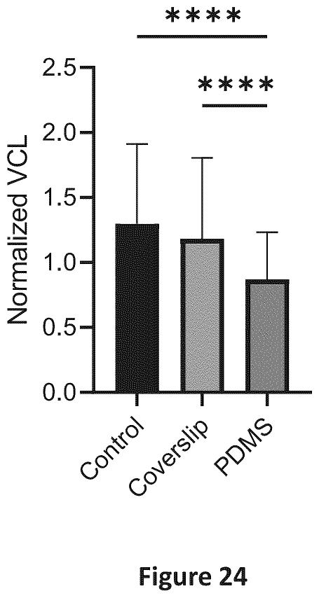

Referring to FIGS. 22 and 24, in an alternative embodiment, rather than having the applicator module 24 in the form of a PDMS fluid chamber 28, the sample of cells is only provided in an acoustic coupling medium 40, such as an acoustic gel as discussed above, and placed above a glass 42 as seen in FIG. 22. It has been observed, per FIGS. 23 and 24, that placing cell samples 26 above a glass coverslip 42 as described is a more efficient method of performing acoustic energy transfer-resulting in greater performance (higher VCL) than the PDMS fluid chamber 28 coupling method with the same level of power applied. It is to be appreciated that the acoustic coupling medium 40 used with the glass coverslip 42 may be of any transmission gel suitable for ultrasound transmission and coupling, and that the glass coverslip 42 may be a disposable, sterile coverslip made from any suitable material that allows transmission of ultrasound waves. It is also to be appreciated that in some embodiments the glass coverslip 42 is not required and the sample is placed directly on the apparatus 10 with acoustic coupling medium 40.

In one embodiment, the acoustic energy emission module 20 is configured to generate ultrasound energy by outputting ultrasound waves within a frequency range of about 15 MHz and about 120 MHz, or by outputting ultrasounds waves at any one of the following non-limiting frequencies from the above range: about 15 MHZ, about 16 MHZ, about 17 MHz, about 18 MHZ, about 19.28 MHz, about 20 MHz, about 25 MHz, about 48.5 MHz, about 75 MHz, about 100 MHZ, and about 120 MHZ.

In some embodiments, the acoustic energy emission module 20 is also configured to access and control the power level of the ultrasound energy output so as to limit the ultrasound waves generated by application of an electrical input power to the IDTs of between about 250 mW and about 3,500 mW, or by setting the electrical input power level to any one the following non-limiting levels from the above range: about 250 mw, about 500 mW, about 750 mW, about 1,000 mW, about 1,250 mW about 1,500 mW, about 1,750 mW, about 2,000 mW, about 2,250 mW, about 2,500 mW, about 2,750 mW, about 3,000 mW and about 3,500 mW.

The IDTs used to generate ultrasound surface acoustic waves in the experiments were fabricated and configured as follows. The emission module 20 was configured to operate at ultrasound frequencies of 19.28 MHz, 48.5 MHz and 100 MHz, and the module 20 comprised two sets of IDTs, with 8, 10 and 2 finger pairs respectively (designed for optimum s11; matched at 50 (2 impedance). The IDTs were fabricated by applying a positive photoresist using standard UV-photolithography on a 0.5 mm, single side polished, 128° Y-cut, X-propagating lithium niobate. Using e-beam evaporation, the LN substrate was coated with 10 nm titanium adhesive layer, 160 nm gold conductive. Subsequently, the excess metal and photoresist was removed with the assistance of lift-off technology. An additional 250 nm silicone dioxide layer was coated on top for protection. The master mould for the polydimethylsiloxane (PDMS) fluid chamber was 3D-printed (Objet Eden 260V) and soaked in 1 M NaOH solution for one hour to remove the supporting materials, following which, a layer of silane (Sigma-Aldrich, Missouri, USA) was assembled on the surface of the mould for easier detachment after PDMS casting. The pre-mixed PDMS (SYLGARD® 184, Dow Corning, with 1:5 mixing ratio of curing agent and polymer) was cast over the mould and degassed. Finally, it was placed on a 70° C. hotplate overnight to obtain the fully-set PDMS fluid chamber.

Details of the IDTs used in the experiments are as follows:

| Frequency | Aperture | S11 | Finger pairs |
|---|---|---|---|
| 19.28 MHz | 2.62 cm | 0.423 | 8 |
| 48.5 MHz | 2.62 cm | 0.523 | 10 |
| 100 MHz | 2.62 cm | 0.414 | 2 |

Further, in some embodiments, either the acoustic energy emission module 20 or the applicator module 24 is configured to control the duration of the ultrasound wave exposure. Preferably, a treatment area, or a sample of the motile cells is exposed to the ultrasound energy for a duration (for example, fixed or pulsing) of between about 5 seconds and about 35 seconds, or for any one of the following non-limiting durations from the above range: about 5 seconds, about 10 seconds, about 15 seconds, about 17 seconds, about 18 seconds, about 19 seconds, about 20 seconds, about 21 sections, about 22 seconds, about 23 seconds, about 24 seconds, about 25 seconds, about 27 seconds, about 30 seconds, and about 35 seconds. The ultrasound energy exposure can be continuous for the period defined or may be delivered in multiple bursts of ultrasound energy, such as 2, 3, 4, 5, 6, 7, 8, 9, 10 or more short bursts of ultrasound energy, which in total amount to the same level of exposure referred to above. The short bursts may be of the same or different duration, and may for example be of about 0.1 second to about 20 seconds, such as about 0.2 to about 15 seconds, about 0.3 to about 10 seconds, about 0.4 to about 8 seconds or about 0.5, 0.8, 1, 1.5, 2, 3, 4 or 5 seconds to about 9, 10, 11, 12, 13 or 14 seconds in duration. The bursts of ultrasound energy may, for example, be separated by a break in exposure that is the same or different to the period of exposure, and the break in exposure can, for example, be of duration as for the periods of exposure exemplified above.

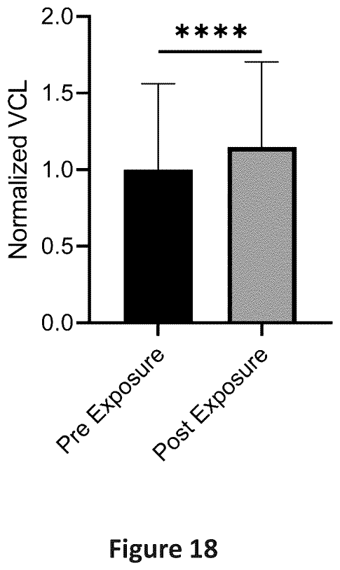

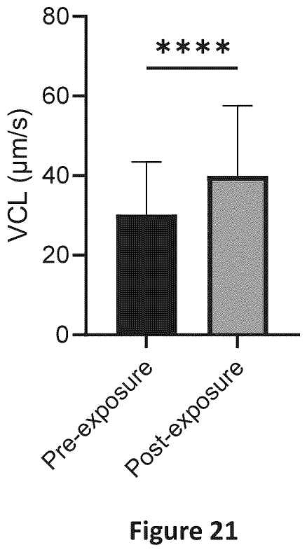

As discussed in relation to the experimental data and discussions below, it has been found unexpectedly, in at least one example, that ultrasound energy having a frequency of about 19.28 MHz combined with an exposure duration of about 20 seconds yielded statistically significant improvement in an increase of average sperm curvilinear velocity (VCL) to the order of 30% over a control group in relation to bull sperm samples, and that the improvement had a sustained effect for about 10 minutes post-exposure. More specifically, it has been observed that motility improvements (VCL) in motile cells ranged between 17.5% and 48.7% over the control groups, with an average motility improvement of 34.4%. Improvements to motility (VCL) has also been observed when ultrasound energy according to parameters of the present invention was applied to a sample of human sperm cells (see FIG. 18). The result demonstrates an improvement of 15% on human sperm VCL post-exposure after treating with ultrasound at 19.28 MHz with electrical input power of 2W for 20s. Referring to FIG. 21, in further experiments where the specific ultrasound parameters were applied to a sample of human sperm cells placed above the glass coverslip in an ultrasound transmission gel, it was observed that the curvilinear velocity (VCL) and straight line velocity (VSL) of the sperm cells improved by 32% and 70%, respectively.

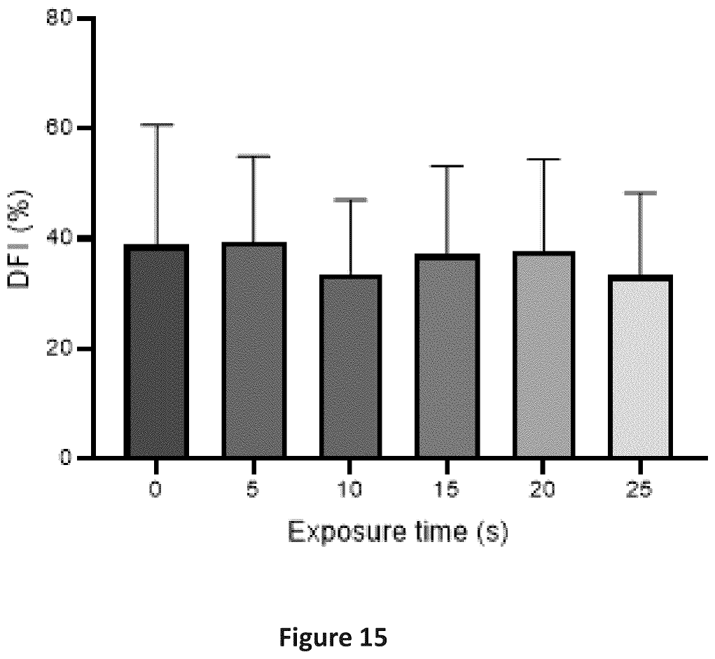

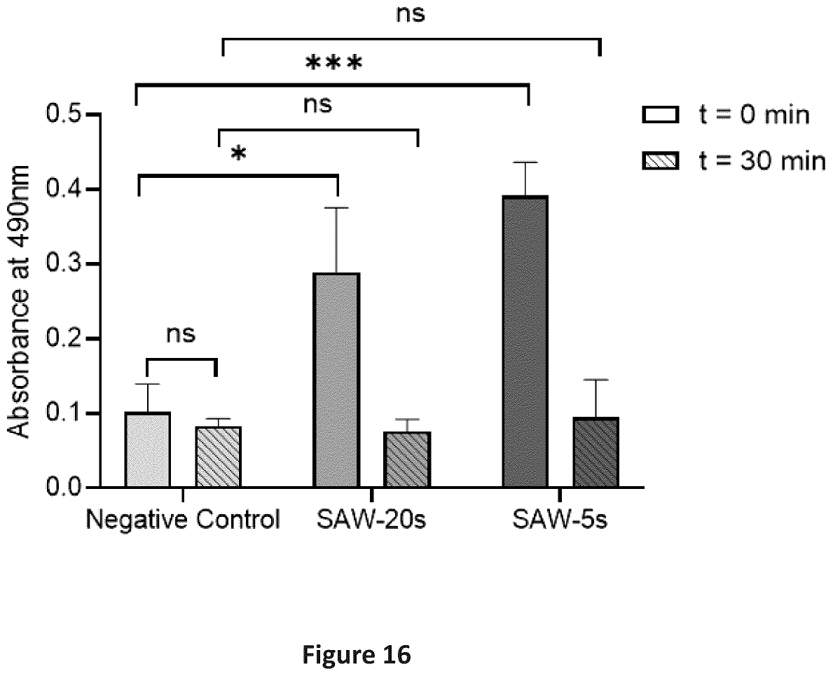

It has also been observed that sperm cells exposed to ultrasound energy according to parameters of the present invention experienced an increase in metabolic activity for a period of about 30 minutes after ultrasound energy exposure. Due to the relatively low selected exposure durations (20 seconds), adverse effects on the motile cells appear to have been negated (see FIGS. 15 and 16 showing no significant changes to cell viability and DNA fragmentation of the exposed sperm cells). It is to be noted that the combination of selected ultrasound parameters produced results that contradicted popular belief and common general knowledge about the use of ultrasound energy in relation to promoting the motility of motile cells and sperm cells. This non-invasive approach to improving motility of motile cells can advantageously be used in vivo and in in vitro fertilisation techniques to improve fertility potential, and specifically in the treatment of asthenozoospermia.

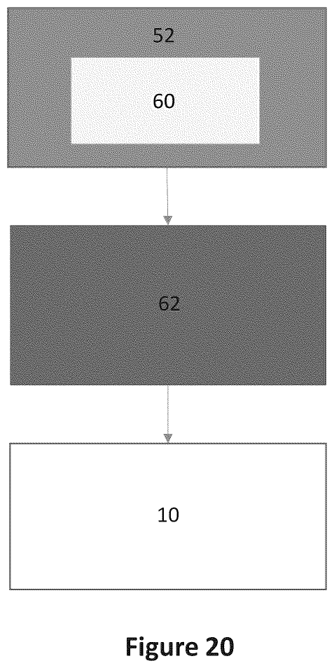

It is to be appreciated that while the example apparatus as provided in FIG. 1 is suitable for exposing motile cells to ultrasound energy in vitro, other forms of the apparatus (such as in the form of a hand-held device) may be suitable for in vivo applications. Modifications may be made to incorporate the apparatus in a hand-held or alternative form suitable for in vivo applications over an anatomical area of a person or animal or the locus of motile cells, without departing from the spirit of the invention. In some embodiment, the apparatus comprises a portable ultrasound generator that is connectable with a hand-held applicator for applying ultrasound energy within the desirable ranges specified by the invention over an anatomical area of a person or animal or the locus of motile cells. The ultrasound generator may be provided with a chargeable battery and configured with suitable digital circuitry and acoustic transducers to generate ultrasound energy. In other embodiments, as seen in FIGS. 19 and 20 for example, the ultrasound generator 10 may be embodied in a portable unit 50, complete with power, control, display and computing systems, that is designed to be used by laboratory technicians in clinic settings. It is to be appreciated that, in some embodiments, the sub-systems for the portable unit 50 may include an onboard computing system 52, a signal generator 54, a signal amplifier 56, a display screen 58, the ultrasound apparatus 10 for providing ultrasound energy to cell samples, and associated cooling systems 51 such as heatsinks, fans and shrouds. In some configurations, with reference to FIG. 20, the computing system 52 is configured to include a therapy planning module, which comprise information about the operating conditions (such as the acoustic frequency, magnitude of acoustic, and acoustic exposure time). The computing system 52 is configured to output data to a controller 62, which in turn controls the ultrasound apparatus 10.

It is also to be appreciated that while surface acoustic waves (SAW) have been described in relation to the example apparatus provided, other non-SAW forms of ultrasound waves, such as bulk acoustic waves (BAW), output in the same power level and frequency is also suitable without departing from the invention.

The invention will now be described further with reference to the following experimental section.

### Materials and Methods

Below is a discussion of the materials and methods employed in conducting the experiments which support the biological efficacy of the present invention.

### Sample Preparation

A cryogenically preserved bull (or human) semen sample was stored in 200 μL vials and thawed in 37° C. water bath for at least five minutes prior to extraction with an artificial insemination syringe. HEPES-based salt buffered solution (NaHCO₃ (4 mM), KCL (5.3 mM), NaCl (117 mM), CaCl₂) (2.3 mM), Na₂HPO₄·2H₂O (0.8 mM), MgSO₄ (0.8 mM), phenol red (0.03 mM), D-glucose (5.5 mM), Na pyruvate (0.33 mM), and Na lactate (21.4 mM)) supplemented with PVA (poly(vinyl alcohol)) (1 mg/mL) to prevent cell adhesion was mixed with the semen sample at a ratio of 10:1 for sperm motion analysis post-ultrasound exposure. To quantify sperm motility and vitality, 10 μL of propidium iodide and 10 μL of 50-fold diluted SYBR14 in DMSO (LIVE/DEAD™ Sperm Viability kit, ThermoFisher) were added to fluorescently label dead and live sperm respectively.

### Sperm Motility Analysis

The SAW device 10 was mounted on a customized microscopic stage and 35-40 μL of sperm sample was evenly distributed in the fluid chamber placed in between two IDTs 20. The IDT 20 pair were actuated with a signal generator (BelektroniG F20 Power Saw, Freital, Germany) to generate a standing surface acoustic wave field. To quantify sperm motility, an image sequence of fluorescently labelled sperm motions at the same imaging field was recorded on chip before and after SAW generation with a 5-MP C-mount PixeLink camera (PL-B872CU, Ottawa, Canada) equipped on an upright microscope (Olympus BX43, Tokyo, Japan) under green fluorescence light excited at the wavelength of 488 nm. The recorded videos were then converted and processed in ImageJ with the OpenCASA plugin, where, only parameters of sperm tracked for 20-99 frames were included in the dataset. Sperm motility parameters were reported as VCL (curvilinear velocity), VAP (average path velocity), VSL (straight line velocity), LIN (linearity) and motility percentage.

### Sperm Vitality Analysis

Post ultrasound exposure at the desired parameters discussed above, the fluid chamber 24 was removed from the top surface 22 of the SAW device 10 and fluorescently labelled sperm samples using sperm live/dead assay (ThermoFisher) was transferred into a haemocytometer (Paul Marienfeld Gmbh & Co. KG, Germany). The images under bright field, green fluorescence excited at the wavelength of 488 nm, and red fluorescence excited at the wavelength of 514 nm were captured and vitality was reported as the percentage of live sperm in total sperm population.

### Sperm DNA Integrity Analysis

Sperm DNA integrity is evaluated with sperm chromatin dispersion test using SpermFunc R DNAf (BRED Life Science Technology Inc, Shenzhen, China). 60 μL of diluted semen sample with concentration from 5-10 million cells per millilitre was dispensed in the dissolved gel which was incubated at 80° C. for 20 min and balanced at 50° C. at least 5 minutes prior to utilisation. 30 μL of the mixture of semen sample and gel was immediately dispensed on each well on the pre-coated slide was placed at the 2-8° C. fridge for at least 5 minutes until the mixture solidified. The slide was then immersed in solution A for 7 minutes exactly, incubated with solution B for 25 minutes under room temperature and horizontally into the tray filled with distilled water for 5 minutes. The slide was then vertically introduced to the slide barrels containing increasing concentration of ethanol (70%, 90% and 100%) for 2 minutes. After air-drying, 15-20 drops of Wright's stain were dispensed on the slide supplemented with 30-40 drops of the Wright's buffer for at least 30 minutes. At least 100 sperms were observed with a colour camera (INFINITY3-3UR, Lumenera) equipped on a general microscopy (Nikon Eclipse Ts2, Tokyo, Japan) under 40× magnification.

### Sperm Metabolic Activity Analysis

Post-ultrasound exposure at desired parameters discussed above, 10 μL of the sperm cells (out of 35-40 μL of sample) were carefully transferred from the device into a 96-well-plate containing 90 μL of media and 10 μL of Cell Counting Kit-8 (CCK-8, Sigma-Aldrich) solution. The solution contains water soluble tetrazolium salt WST-8 [2-(2-methoxy-4-nitrophenyl)-3-(4-nitrophenyl)-5-(2,4-disulfophenyl)-2H-tetrazolium, monosodium salt] that is converted into a formazan dye by living cells and therefore the change in the absorbance correlates with the metabolic activity when the number of cells is same in all samples. The cells were incubated immediately after ultrasound treatment in humidified incubator at 37° C. with 5% CO₂ for 1 h. A second set of experiments was performed with sperm cells incubated in only media after ultrasound treatment for 30 min, followed by the addition of CCK-8 solution and incubation for another 1 h. A negative control group, where the cells were not exposed to ultrasound. At the end of 1 h, the absorbance was recorded using a multi-plate reader (Multiskan™ FC Microplate Photometer, Thermofisher Scientific) at 490 nm wavelength. Media which did not contain cells was used as blank for the absorbance calculations.

Intracellular H₂O₂ Analysis

Intracellular H₂O₂ was analysed using a fluorescent sensor (MAK164, Sigma-Aldrich) with a green fluorescence excited at 490 nm and emission at 520 nm, according to the manufacturer's instructions with slight modifications performed in order to analyse suspension cells. The sensor working solution was prepared by mixing fluorescent peroxide sensor solution with the assay buffer in the ration of 1 μL to 500 μL, respectively. Post-ultrasound exposure, 10 μL of the sperm cells (out of 35-40 μL of sample) was added to 100 μL of the sensor working solution and incubated at room temperature for 30 min. The sensor solution was removed by centrifugation (spinning for 5 min at 200 relative centrifugal force (rcf)) followed by the resuspension of cells in 20 μL of media. The cells were then transferred into a 96-well plate and fixed by the addition of 80 μL of ice-cold methanol at −20° C. The fixed cells were washed with phosphate-buffered saline (PBS) for three times and imaged using a Nikon Eclipse Ts2 fluorescence microscope with CFI Plan Fluor 20×/0.5 NA objective. A group of cells was incubated with 100 μM of H₂O₂ in media for 30 min as positive control and the cells not exposed to SAW was labelled as negative control. The fluorescence was reported as the average fluorescence intensity (in the form of arbitrary units, a.u.)/cell acquired from 4 wells that contained at least 2000 cells in each group.

### Statistical Analysis

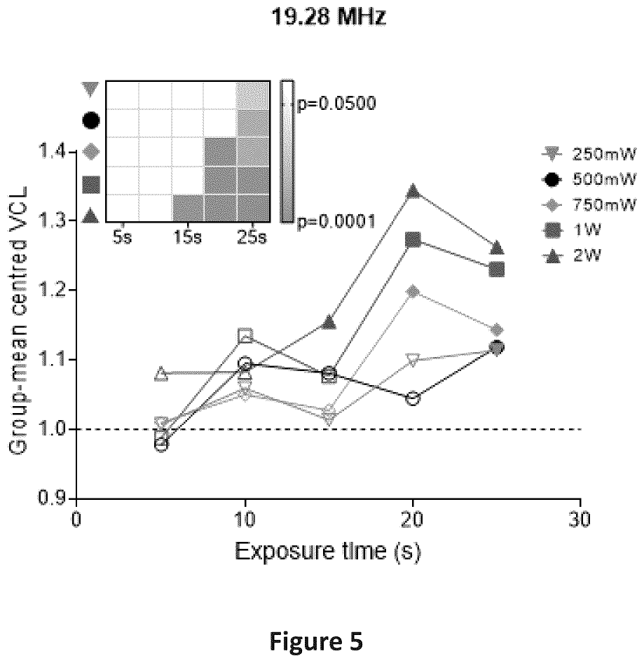

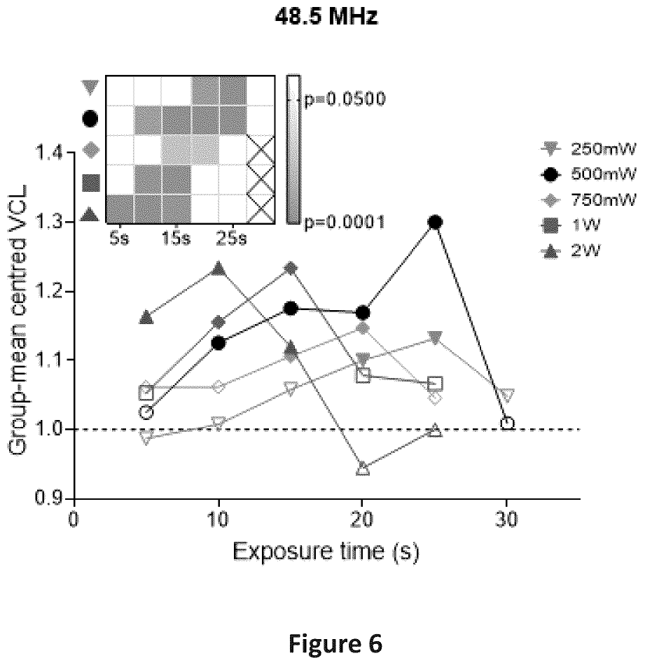

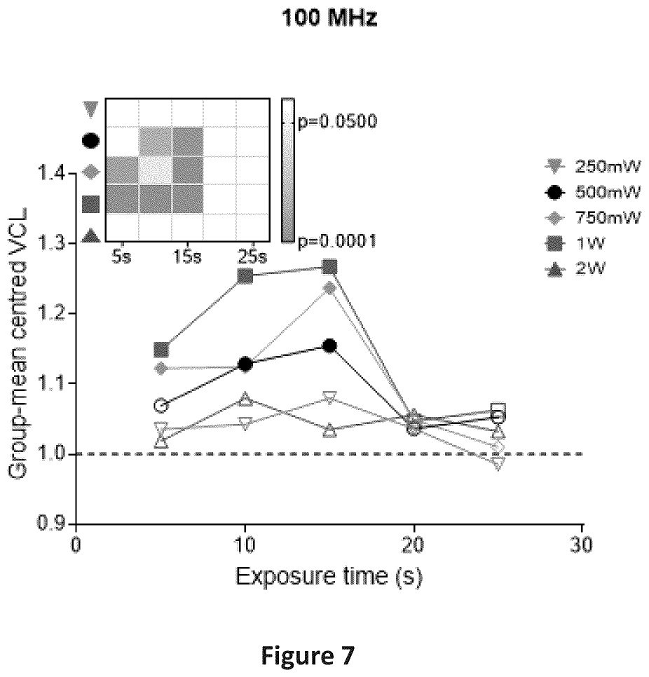

Post-exposure sperm motility parameters (i.e. VCL, VAP, LIN and motility percentage) and respective pre-exposure parameters of each sperm analysed were group-mean centred to its corresponding pre-exposure (i.e. direct control) quantity (see FIGS. 5-13). Each sample was group-mean centred at pre-exposure levels to average their starting level comparable, providing a basis for fair comparison, accommodating for the heterogeneity in raw sample examined. Two-way ANOVA with post-hoc Bonferroni corrections was used to analyse the validity of exposure conditions by comparing the post-exposure parameters with its pre-exposure counterpart over the range of exposure conditions considered. The performances across different exposure times were analysed using the same statistical tests via comparing group-mean centred post-exposure motility parameters at each exposure condition.

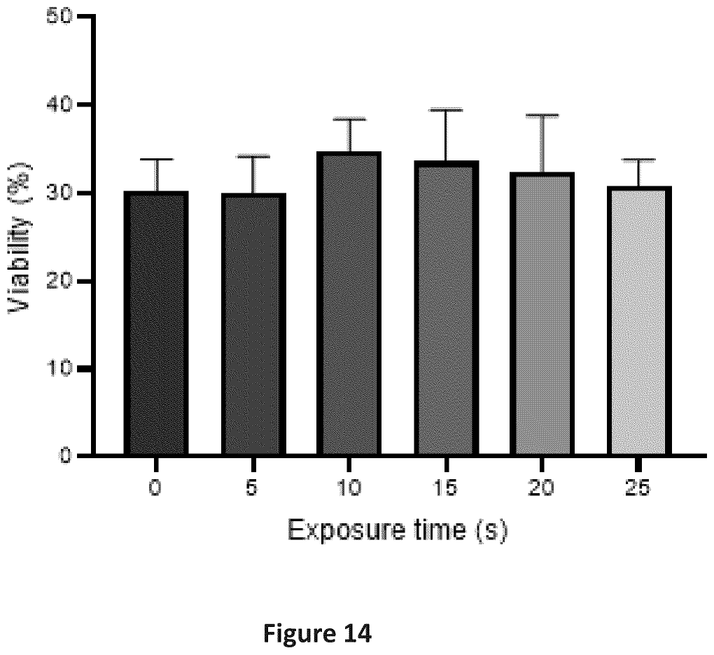

Statistical analysis for sperm viability, DNA integrity, metabolic activity and intracellular H₂O₂ studies (FIGS. 14-17) were performed using One-way ANOVA with post-hoc Bonferroni corrections.

### Experimental Results

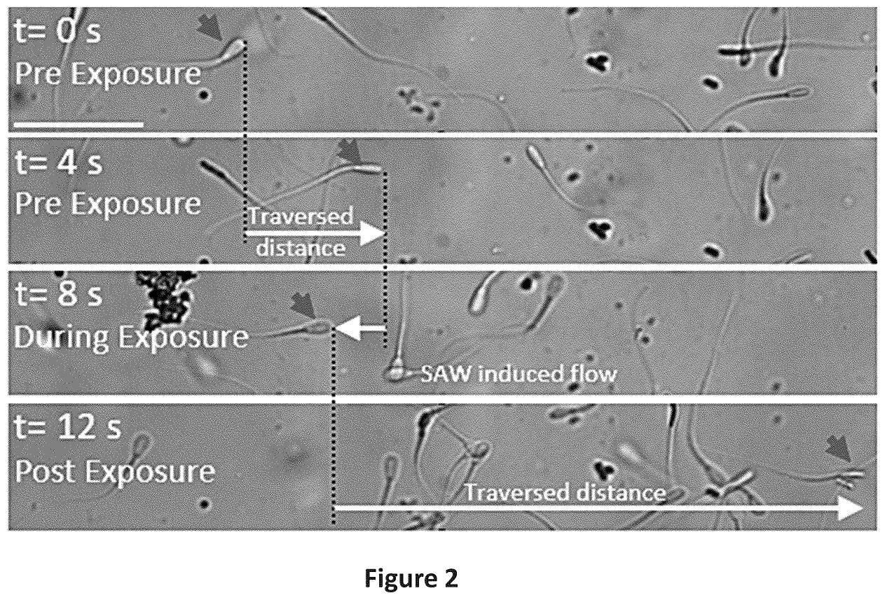

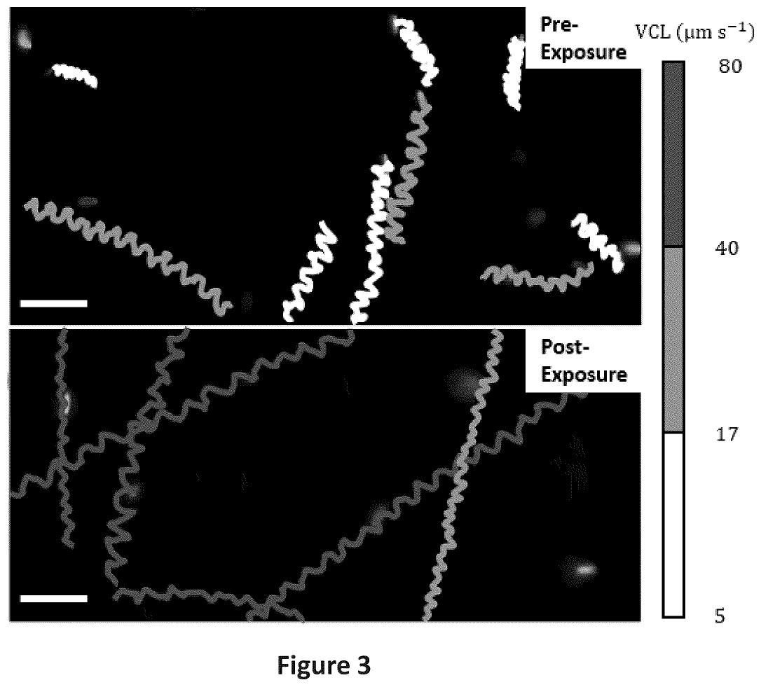

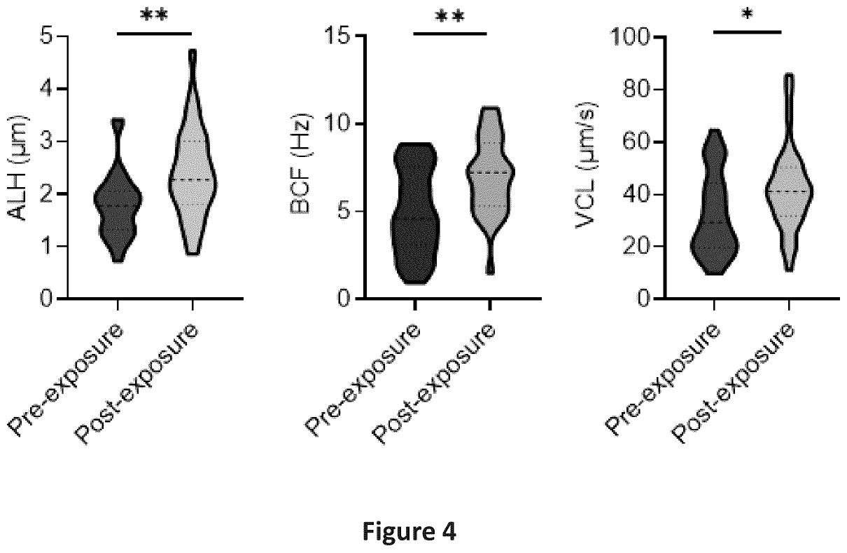

Referring to FIG. 2, ultrasonic exposure at 19.28 MHz and 2 W was observed to maximally increase the distance travelled by an individual sperm per unit time—an effect which persists post-exposure. FIG. 3 depicts the swimming trajectories of sperm pre—and post-exposure, with the colour of trajectories corresponding to the average swimming velocity of sperm along its instantaneous trajectory (curvilinear velocity). As can be seen from FIG. 4, the increase in the swimming velocity post exposure is related to an increase in both lateral head displacement (ALH) and beat cross frequency (BCF), indicating that acoustic exposure influences the fundamental characteristics of sperm beating behaviour (FIG. 1d, e). Specifically, ALH increased by 34% (n=30, N=3, P=0.0006) and BCF increased by 42% (n=30, N=3, P<0.0001) post-exposure. It appears that several factors might contribute to regulate the beating behaviour, including an increase in the rate of metabolic activity, regulation of mechano-sensitive ion channels that influences the flagellar waveform, and a change in the stiffness of the cell membrane that can influence the rate of energy dissipation into the fluid.

Referring to FIGS. 5-7, the group-centred mean VCL was quantified for a range of ultrasonic frequency (19.28 MHz, 48.5 MHz and 100 MHZ), input electrical power (250 mW to 2,000 mW) and exposure time (5 seconds to 25 seconds). For each data point in these plots, the significance of the normalised VCL has been found using a two-way ANOVA with post-hoc Bonferroni corrections. The p-value obtained is colour coded against the exposure condition in the inserted panels. Furthermore, in the main panel, a condition which resulted in a p-value <0.05 is shown using a solid (rather than outline) marker. FIG. 5 shows the group-centred mean post-exposure VCL for 19.28 MHz over a range of exposure times (5 s to 25 s) and input electrical power (250 mW to 2 W). For 20 s of exposure at 2 W, the maximum increase in sperm VCL was achieved, resulting in an increase in VCL of 34%, with post-exposure VCL resulting in a p-value <0.05 when compared with its corresponding pre-exposure values (FIG. 5). In contrast, shorter exposure times at this power level were not sufficient to generate a stable and consistent increase in sperm VCL, reflected by the statistically insignificant result. The range of exposure times required to achieve a significant increase in VCL is most clearly seen by examining the shaded regions in each panel's insert. For 19.28 MHz there is a lower cut off in exposure time, below which the VCL change is not significant, perhaps unsurprisingly, this cut off occurs at a larger exposure time for lower powers. For the case of 48.5 MHZ, an upper limit to the exposure range is also observed beyond which the VCL change loses significance. Again, this exposure time limit is larger for lower power levels.

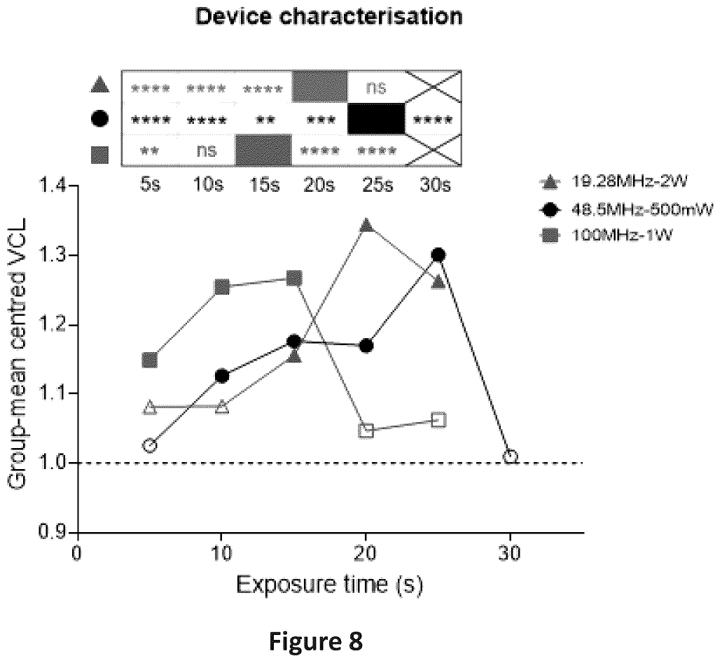

The data in FIGS. 5 to 7 established the VCL boost which can be obtained for each exposure time, power and frequency, and shows a range over which the VCL change is statistically significant. However, this is insufficient to statistically establish the trend in VCL change over a range of exposure times, and thus, information that is required to select the optimum excitation conditions within the tested range. To address this, FIG. 8 consolidates the operating power conditions at each frequency which resulted in the highest increase in the post-exposure VCL. In this panel the insert shows the statistical significance in the change of VCL across exposure times, with reference to the highest value obtained for that given power and frequency. The significances are established using by comparing group-mean centred post-exposure VCL through further use of the two-way ANOVA with post-hoc Bonferroni corrections. Hence, for 48.5 MHz exposure at 500 mW, an exposure of 20 s gives a VCL increase which is statistically significantly higher than both shorter and longer exposure times. Whilst, for the highest VCL change condition of 19.28 MHz, 2 W, the results of an exposure of 20 and 25 s are not significantly different.

Overall, the data shown in FIGS. 5 to 8 shows that the average VCL of a sample of sperm can be increased by ultrasound when using a suitable combination of frequency, power and exposure time. Furthermore, whilst low acoustic energy may be insufficient to generate statistically effective VCL boost, if the exposure is prolonged further, there is a possible onset of negative effects. The deleterious nature of over exposure could potentially be due to the induced mechanical stresses. It seems excessive stresses cause increased mitochondrial membrane permeability and interrupted ATP production, and that ATP availability is directly linked to sperm motility. Moreover, prolonged mechanical stimulation has been shown to increase membrane stiffness in adherent cells, and a similar effect to reduce membrane fluidity and increase cell rigidity can contribute to reduced sperm motility levels.

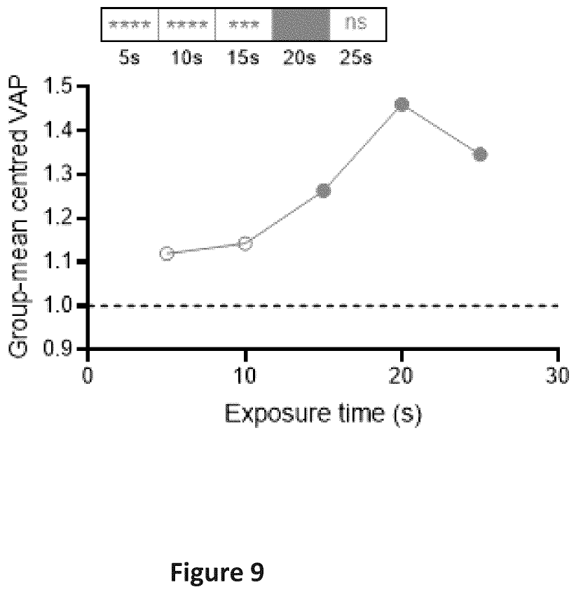

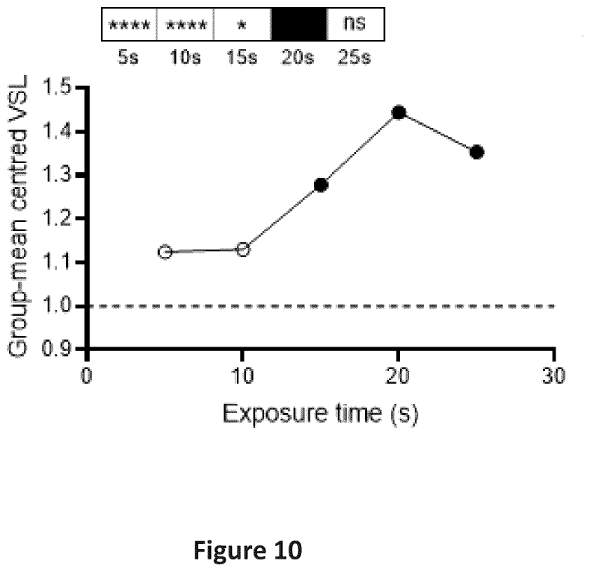

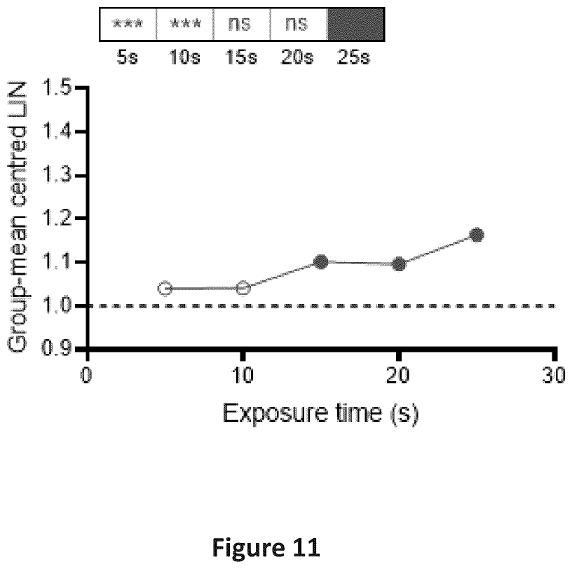

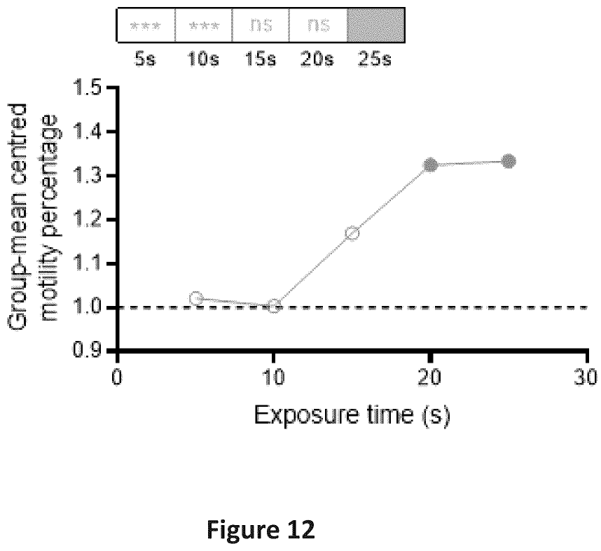

In addition to the actual swimming velocity of the sperm (VCL), other parameters are typically considered to describe a sperm's motion. Sperm motility parameters also include the percentage of sperm exhibiting swimming behaviour in the semen sample and the normality of sperm movement, the latter being characterized by average path velocity (VAP: swimming velocity along the time-averaged trajectory, FIG. 9), straight line velocity (VSL: time average velocity along the straight line between the start and final detection points, FIG. 10), and linearity (LIN=VSL/VCL; FIG. 11). FIGS. 9-12 details these additional sperm motility parameters contrasting pre—and post-exposure using a frequency of 19.28 MHz and power of 2 W. The statistical approach taken is similar to FIGS. 5-8, the relative increase in each parameter for individual excitation conditions are established, and a solid marker is used for significant changes registered, and to examine the trend across exposure times, an insert is included which shows the significantly different data points with reference to the exposure which yields the highest group-centred mean value. After 20 s of exposure (identified as the optimum exposure time by examination of representative probability distribution functions of VCL of each tracked sperm in individual experiments, shown in supplementary information FIG. S2), the sperm swim faster, exhibiting 46% increase in VAP and 44% increase in VSL. It is noteworthy that the percentage increase of both VAP and VSL were higher than VCL, this is mainly due to a 10% increase in LIN. The results indicate that ultrasonic exposed sperm not only exhibit an increased instantaneous swimming velocity, but also swim along a straighter path (as was indicated by the images in FIG. 3). Such straighter motion has previously been linked to a decrease in the level of bend asymmetry in the flagellar wave. Furthermore, we observe that the percentage of motile sperm increased by 33% for the longest exposure time (FIG. 12) among the live sperm population in the sample. This increase in the number of motile sperms is also attributed to the observed effect of ultrasonic exposure rendering immotile sperm, motile. Sperm motility is a significant predictor of male fertility potential and is directly linked to fertilization success, suggesting the improved performance of ultrasonic exposed sperm to be conducive for in vivo and in vitro fertilization. It is therefore to be appreciated that metabolic activities induced by the ultrasound exposure according to the specified parameters resulted in improved mechanical motility of the cells.

To test the biocompatibility of the discussed method, sperm vitality and DNA integrity pre—and post-ultrasonic exposure (19.28 MHZ, 2 W) were compared (FIGS. 14-15). No significant changes in sperm live/dead were observed as shown in FIG. 14, indicating that spermatozoa preserve their membrane integrity, even after the 20 s of acoustic exposure, which is required to achieve the highest relative increase in VCL (FIGS. 5-8). Similarly, the DNA fragmentation index (i.e., the percentage of sperm with fragmented DNA) remained unchanged by ultrasonic exposure, demonstrating that the ultrasound exposure method as discussed does not have an untoward impact on interrupting sperm intracellular DNA structures or increase DNA damage level. Sperm DNA integrity is an important indicator of male infertility, and strongly correlated with the success rate of natural and the outcome of assisted reproduction. Combined with the significant effect of enhanced sperm motility and the lack of adverse effects associated with biocompatibility imposed by ultrasonic exposure, our results demonstrate the significant potential to boost sperm motility, benefitting patients with male infertility issues in either natural reproduction or assisted reproductive technology.

### DISCUSSION

Sperm motility is produced by sperm flagellar beating, and an improvement in sperm motility is associated with the rate of energy production and its utilization by the sperm flagellum. The sperm metabolic process is responsible for intercellular energy production where ATP is generated through electron transportation from NADH to oxygen via NADH dehydrogenase protein. ATP hydrolysis releases energy to drive the dynein molecular motor in the 9+2 structure of the flagellar axoneme. Therefore, a potential change in rate of energy production for flagellar activity can influence sperm motility.

The results show that high frequency (19.28 MHZ) ultrasonic exposure at 2 W for 20 s boosts sperm swimming velocity without imposing adverse effects on either their viability or DNA integrity (FIGS. 14-15). Potentially, this increase in swimming performance could be due to an alteration in the regulation of bioenergetic mechanisms controlling flagellar activity. Flagella bending occurs as a result of the activation of AAA domains on axonemal dyneins. At these sites, chemical energy from ATP hydrolysis is transduced to mechanical energy causing the sliding of the outer axonemal doublet microtubules, resulting in a rhythmic beating of the flagellum. To probe potential alterations to this process as a result of ultrasonic exposure, the rate of metabolic activity was assessed using an MTS assay to examine the sperm metabolic rate immediately post-exposure and 30 minutes post-exposure. Sperm cells exposed to ultrasound (19.28 MHz, 2 W) for 5 s and 20 s demonstrated a significantly higher rate of metabolic activity than the negative control of un-exposed sperm cells (FIG. 16). The effect on sperm metabolic activity is reversible and a return to a normal range observed 30 minutes post-exposure. This ultrasound induced increase in cell metabolic activity could act as the driving mechanism to boost sperm motility.

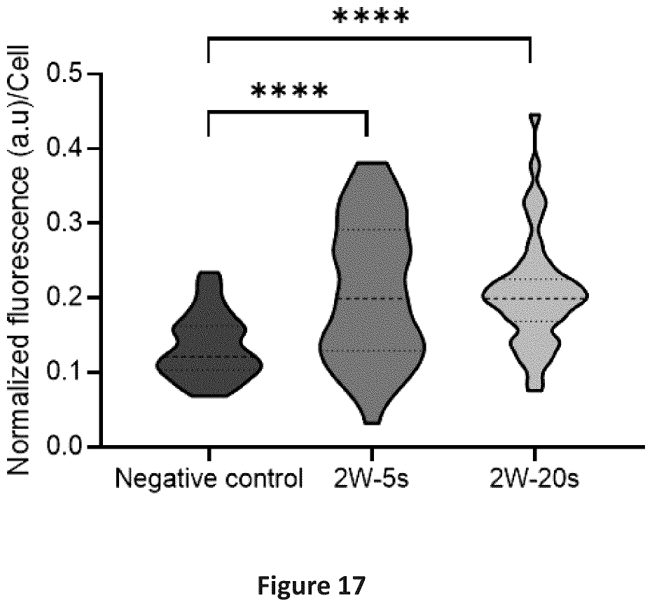

An increase in metabolic activity in sperm can be linked directly to a higher rate of energy production. In sperm, 0.2-2% of the O2 involved in the mitochondrial respiratory chain, where NADH and succinates are oxidized to generate energy for ATP synthesis, escapes and produces ROS. Hence, increased metabolism inevitably results in a higher level of ROS production in the cell. Whilst low to moderate levels of ROS can regulate physiological functions, at high concentrations, the effects can be detrimental. ROS at high concentrations causes decrease in both sperm motility and DNA integrity. To examine this, we measured the level of H₂O₂ in the cells, this being a common form of endogenous ROS. A higher concentration of intracellular H₂O₂ was found in exposed cells (benchmarked against un-exposed cells; negative control), as shown in FIG. 17. However, with increased ROS production post-ultrasonic exposure, no significant changes were witnessed for sperm viability and DNA integrity (FIGS. 14-15), indicating the increased level of ROS here was not adequate to damage to sperm DNA and functionality.

This increase in metabolism and sperm motility post ultrasonic exposure might be attributed to effects on cell signalling pathways. An influx of Na⁺, H⁺, Ca²⁺ and/or HCO₃ can cause an increase in sperm motility, due to the activation of ion channels. Ultrasonic exposure has been linked to the change of cell membrane permeabilization accompanied by the cell dependent influx of Ca²⁺ through voltage gated calcium channel over a range of frequencies. In sperm cells, Ca²⁺ influx is also reported to regulate a sperm's swimming pattern. However, our results show a sperm motility boost post-ultrasonic exposure occurs even when suspended in a buffer absent of Ca²⁺, seemingly ruling out the role of extracellular effects. Sperm does not only obtain Ca²⁺ from the external environment. The intracellular Ca²⁺, stored in the base of flagellum, can propagate down the flagellar midpiece upon activation, this might also play a role in motility enhancement. Furthermore, studies show elevation of Ca²⁺ along flagellar induces increased amplitude of the principle flagellar bend on one side, producing a highly asymmetrical swimming pattern, known as sperm hyperactivation, which is a swimming mode prior to capacitation. Indeed, across the various research on the regulation role of Ca²⁺ on sperm motility, the change in Ca²⁺ concentration is typically accompanied with sperm hyperactivation, regardless of the source of Ca²⁺. Compared with a sperm's normal swimming pattern, hyperactivation swimming shows increased swimming velocity and flagella amplitude, but decreased linearity and level of flagella wave bend symmetry. We note that post ultrasonic exposure, an increase in the linearity of sperm motion is observed.

For natural reproduction the number of motile sperm in the sample is directly linked to fertilization success, and in the case of assisted reproduction, the number of motile sperm in the processed sample is also important for the treatment method selection. For example, intrauterine insemination (IUI), involving the injection of a prepared sperm sample directly to female uterus, is recognized as an economical and effective approach as it removes the need for egg retrieval, but requires a high number of motile sperm (˜5 million). Whilst, in vitro fertilization (IVF) involves incubating selected sperm with an egg in a Petri dish, usually requiring a sample of over 50,000 motile sperm with an average velocity in excess of 50 μm s⁻¹ in order to achieve the best fertilization rate⁶⁹. Based on the importance of sperm motility in the employment of both IUI and IVF, our findings offer the possibility of using high frequency ultrasound as a non-invasive and efficient method to increase sperm motility, thus, potentially circumventing the need for intracytoplasmic injection (ICSI), where the invasive procedures frequently cause detrimental effect for zygote and offspring health, providing more opportunities to enhance fertilization success rates.

It will be understood that the invention disclosed and defined in this specification extends to all alternative combinations of two or more of the individual features mentioned or evident from the text or drawings. All of these different combinations constitute various alternative aspects of the invention.

In the description and drawings of this embodiment, same reference numerals are used as have been used in respect of the first embodiment, to denote and refer to corresponding features.

While various embodiments of the present invention have been described above, it should be understood that they have been presented by way of example only, and not by way of limitation. It will be apparent to a person skilled in the relevant art that various changes in form and detail can be made therein without departing from the spirit and scope of the invention. Thus, the present invention should not be limited by any of the above described exemplary embodiments.

1. An apparatus for use in promoting motility of flagellar cells, comprising an acoustic energy emission module configured to generate ultrasound energy, the module being configured to generate ultrasound waves within a frequency range of about 2 MHz and about 120 MHz; and an applicator module configured to direct the generated ultrasound waves to a locus of the cells for a duration of between about 5 seconds and about 35 seconds.

2. The apparatus of claim 1, wherein the acoustic energy emission module comprises two sets of interdigitate transducers configured to generate ultrasound energy in the form of standing acoustic waves.

3. The apparatus of claim 1, wherein the applicator module comprises a polydimethylsiloxane fluid chamber or a fluid chamber attached with acoustic coupling medium to house the sperm cells.

4. The apparatus of claim 3, wherein the fluid chamber attached with acoustic coupling medium is a glass fluid chamber.

5. The apparatus of claim 1, wherein the acoustic energy emission module is configured to generate ultrasound waves within a frequency range of about 5 MHz and about 100 MHz, more preferably of about 10 MHz and about 50 MHz, and yet more preferably of about 19.28 MHz.

6. (canceled)

7. (canceled)

8. The apparatus of claim 1, wherein the applicator module is configured to direct the generated ultrasound waves for a duration of between about 15 seconds and about 25 seconds.

9. The apparatus of claim 8, wherein the applicator module is configured to direct the generated ultrasound waves for a duration of about 20 seconds.

10. A method for promoting motility of flagellar cells, comprising the steps of:
generating ultrasound waves within a frequency range of about 2 MHz and about 120 MHz; and
exposing a locus of the cells to the generated ultrasound waves for a duration of between about 5 seconds and about 35 seconds.

11. The method of claim 10, wherein the ultrasound waves are generated in the form of standing acoustic waves by an acoustic energy emission module comprising two sets of interdigitate transducers.

12. The method of claim 10, wherein the locus of the cells is exposed to the generated ultrasound waves via an applicator module.

13. The method of claim 10, wherein the cells are housed in a polydimethylsiloxane fluid chamber or a fluid chamber attached with acoustic coupling medium for exposure to the generated ultrasound waves.

14. The method of claim 13, wherein the fluid chamber attached with acoustic coupling medium is a glass fluid chamber.

15. The method of claim 10, comprising generating ultrasound waves within a frequency range of about 5 MHz and about 100 MHz, more preferably of about 10 MHz and about 50 MHZ, and yet more preferably of about 19.28 MHz.

16. (canceled)

17. (canceled)

18. The method of claim 10, comprising exposing the cells to the generated ultrasound waves for a duration of between about 15 seconds and about 25 seconds.

19. The method of claim 10, comprising exposing the motile animal cells to the generated ultrasound waves for a duration of about 20 seconds.

20. (canceled)

21. (canceled)

22. The method of claim 10, wherein the cells are sperm cells, for treating asthenozoospermia, for improving fertilisation potential of sperm cells or for determining sperm cell viability.

23. (canceled)

24. The method of claim 10, wherein an acoustic energy emission module is provided to output ultrasound at a given frequency range, input power and for a particular duration based on cellular type.

25. The method of claim 10, wherein improvements in metabolic activity resulting from the cells' exposure to acoustic energy emissions of the applicator module leads to an increase in mechanical motility of the cells.

26. The method of claim 10, wherein ultrasound waves are transmitted to the cells through an acoustic coupling medium and optionally a glass coverslip.

27. The method of claim 10, further providing a computer system for administering the generation and transmission of the ultrasound wave to the cells, wherein the computer system controls variables including frequency of the ultrasound waves, magnitude of ultrasound waves and ultrasound exposure duration.
